(19) United States
(12) Patent Application Publication
(10) Pub. No.: US 2009/0171422 A1
(43) Pub. Date: Jul. 2, 2009

(54) Radiation treatment device

(75) Inventors: W. Daniel Hillis, Encino, CA (US); Leroy E. Hood, Seattle, WA (US); Roderick A. Hyde, Redmond, WA (US); Eric C. Leuthardt, St. Louis, MO (US); Nathan P. Myhrvold, Medina, WA (US); Clarence T. Tegreene, Bellevue, WA (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC , a limited liability corporation of the State of Delaware

(21) Appl. No.: 12/006,272

(22) Filed: Dec. 31, 2007

(51) Int. Cl. A61B 18/18

(52) U.S. Cl. 607/88

(57) ABSTRACT

Embodiments include an apparatus, a medical device, a method and a system. The medical device includes an ellipsoidally-shaped reflector having a first focus and a second focus. The ellipsoidally-shaped reflector also provides a translational coupling of electromagnetic energy from the first focus to the second focus. The medical device also includes a controllable electromagnetic energy source aligned to emit a non-biologically emitted electromagnetic energy in a proximity to the first focus.

### TECHNICAL FIELD

The present application relates to devices, methods and/or systems for treatment and/or management of disease, disorders, or conditions using electromagnetic radiation.

### SUMMARY

An embodiment provides a medical device. In an aspect, the medical device includes an ellipsoidally-shaped reflector having a first focus and a second focus. The ellipsoidally-shaped reflector provides a translational coupling of electromagnetic energy from the first focus to the second focus. The medical device also includes a controllable electromagnetic energy source aligned to emit a non-biologically emitted electromagnetic energy in a proximity to the first focus.

In an embodiment, a medical device comprises of a radiation-collector mirror. In a further embodiment, the radiation-collector mirror may be a birefringence mirror or it may be a light collector-mirror. In a further embodiment, the light-collector mirror collects light from the first and/or second focus and directs the collected light to a biological tissue. In another embodiment, the radiation-collector mirror includes at least one mirror-cooling device. In an embodiment, the medical device further comprises a plurality of beam-splitters. The at least one of the plurality of beam-splitters may produce an adjustable beam cross-section. An embodiment of the medical device further comprises a plurality of polarizers. The plurality of polarizers may polarize electromagnetic radiation either in linear, horizontal, circular or elliptical polarization mode. Furthermore, the at least one of the plurality of polarizers may be configured to function in conjunction with a beam splitter. In an embodiment, the beam splitter may divide an input polarized beam into a first and a second part with a dividing beam ratio which may be continually controllable. In yet another embodiment, the medical device includes a condenser lens system. The medical device may further comprise of a monochromator. Alternatively or additionally the medical device may comprise of an image receiver.

In an alternative embodiment, the ellipsoidally-shaped reflector includes an opening configured to allow positioning of a portion of a body proximate with the second focus. A further embodiment of the ellipsoidally-shaped reflector includes a conductor. In another embodiment, the conductor includes at least one of an aluminum, a tin, a stainless steel, a silver, a gold, a copper, an iron, a carbon, an iridium, an indium, a lead, a magnesium, a nickel, a nichrome, a palladium, a rhodium, a silver, a tantalum, a titanium, a tungsten, a zinc, a platinum and/or a zirconium. In yet another embodiment, the ellipsoidally-shaped reflector includes a dielectric material. In a further embodiment, the dielectric material includes a rubber, a plastic, a porcelain, a ceramic, a mica, a glass, a metal oxide, a perfect vacuum, a dry air, a pure dry gas, a helium and/or a nitrogen.

In an alternative or additional embodiment, the non-biologically emitted electromagnetic energy includes at least one of a visible light, a laser energy, an ultraviolet energy, an infrared energy, an X-ray or a microwave. In an embodiment, the controllable electromagnetic energy source includes a computer configured to control delivery of the non-biological electromagnetic energy. In a further embodiment, the controllable electromagnetic energy source includes at least one of a plano-convex lens, a meniscus lens, a cylindrical lens, a parabolic lens, an acrylic lens, a glass lens, a quartz lens, a Fresnel lens, a Pendry lens, a band pass filter, a polarizer, a dichroic material, a monochromator and/or a collimator. In yet another embodiment, the controllable electromagnetic energy source regulates at least a characteristic of the non-biologically emitted electromagnetic energy. In a further embodiment, the at least a characteristic of the non-biologically emitted electromagnetic energy includes at least one of a wavelength, a frequency, an amplitude, a phase, a polarization and/or a bandwidth. In addition to the foregoing, other embodiments of the medical device described in the claims, drawings, and text form a part of the patent application.

An embodiment provides a method of treating a portion of a living body. The method includes emitting a selected dose corresponding to a predicted therapeutic level of electromagnetic energy in a proximity to a first focus of an ellipsoid. The method also includes translating the selected dose corresponding to a predicted therapeutic level of electromagnetic energy to a second focus of the ellipsoid. The method further includes activating a biological tissue in a proximity to the second focus with the selected dose corresponding to a predicted therapeutic level of electromagnetic energy.

In an embodiment of the foregoing method, the emitting includes emitting electromagnetic energy having at least one wavelength between 1 nm and 700 nm; 2.0 μm and 10 μm; and/or 1 cm and 100 cm. In another illustrative embodiment, the emitting a selected dose of electromagnetic energy includes electromagnetic energy having at least one wavelength between 700 and 2000 nm. In a further illustrative embodiment, the emitting a selected dose of electromagnetic energy includes electromagnetic energy having at least one wavelength between 10 μm and 1 cm. In a further embodiment, the emitting includes electromagnetic energy having at least one of an amplitude variation, a phase variation and/or a variable polarization parameter. In yet another embodiment of the foregoing method, the emitting electromagnetic energy as a series of one or more pulses, each of the pulses having at least a pulse duration between a picosecond and a second.

In a further embodiment of the method, the translating includes the second focus having a volume between 1000 μm³ and 1000 cm³ in a proximity to a biological tissue.

In another embodiment of the foregoing method, the activating a biological tissue includes selectively energizing the first portion of the biological tissue differentially relative to a second portion of the biological tissue. In yet another embodiment, the activating includes coverage of 0.1% to 100% of the biological tissue with the second focus. In an embodiment of the method includes activating the biological tissue using electromagnetic energy having a level between 1 to 100,000 milli Joules per gram of biological tissue at the second focus. In a further embodiment, the activating includes making the second focus at least substantially coincidental with a first portion of the biological tissue and then making the second focus at least substantially coincidental with a second portion of the biological tissue. In addition to the foregoing, other embodiments of the method described in the claims, drawings, and text form a part of the patent application.

A medical device for treating a portion of a living body is provided. The medical device includes a means for emitting a selected dose corresponding to a predicted therapeutic level of electromagnetic energy in a proximity to the first focus of the ellipsoid. The medical device also provides for a means for translating the selected dose corresponding to a predicted therapeutic level of electromagnetic energy to the second focus of the ellipsoid. The medical device provides a means for activating a biological tissue in a proximity to the second focus with the selected dose corresponding to a predicted therapeutic level of electromagnetic energy.

An embodiment provides a system of treating a portion of a living body. The system includes an ellipsoidally-shaped reflector having a first focus and a second focus, and shaped to provide a translational coupling of electromagnetic energy from the first focus to the second focus. The system also includes a controllable electromagnetic energy source aligned to emit a non-biologically emitted electromagnetic energy in a proximity to the first focus. The system further includes an electromagnetic energy source controller coupled to the energy source and having a regulator. The regulator includes electrical circuitry configured to govern at least one of a wavelength, an amplitude, a polarization state, a bandwidth, a collimation filter, a phase shift, a pulse, a frequency and/or a focus. In addition to the foregoing, other embodiments of the system described in the claims, drawings, and text form a part of the patent application.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 illustrates a medical device;

FIG. 2 illustrates an example of a medical device;

FIG. 3 is a perspective view of an embodiment of an ellipsoidally-shaped reflector;

FIG. 4 illustrates an example of operational flow in which embodiments may be implemented;

FIG. 5 illustrates an alternative embodiment operational flow in which embodiments may be implemented;

FIG. 6 illustrates an alternative embodiment of operational flow in which embodiments may be implemented;

FIG. 7 illustrates an alternative embodiment of operational flow in which embodiments may be implemented;

FIG. 8 schematically illustrates a medical device in which embodiments may be implemented;

FIG. 9 schematically illustrates a medical device in which embodiments may be implemented;

FIG. 10 schematically illustrates a medical device in which embodiments may be implemented;

FIG. 11 schematically illustrates a medical device in which embodiments may be implemented;

FIG. 12 schematically illustrates a medical device in which embodiments may be implemented;

FIG. 13 illustrates an example of a medical device that may be used to implement embodiments; and

FIG. 14 illustrates an example of a system that may be used to implement embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The following disclosure is drawn to a medical device comprising an ellipsoidally-shaped reflector having a first focus and a second focus, and providing a translational coupling of electromagnetic energy from the first focus to the second focus. In an aspect, the disclosure is drawn to a medical device comprising a half ellipsoid configured to, and/or structured to at least partially or completely be coupled to a controllable electromagnetic energy source aligned to emit a non-biologically emitted electromagnetic energy in a proximity to the first focus and includes an opening configured to at least partially or completely allow the positioning of at least a portion of a living body or a biological tissue in proximity to the second focus.

In some embodiments, a medical device is a structure comprising a fully or partially enclosed ellipsoid configured to, and/or structured to at least partially or completely be aligned to a controllable electromagnetic energy source aligned to emit a non-biologically emitted electromagnetic energy in a proximity to the first focus and includes an opening configured to at least partially or completely allow the positioning of a portion of an animal body proximate with the second focus.

In other embodiments, a medical device is a structure enclosing a substructure comprising a fully or partially enclosed ellipsoid configured to, and/or structured to at least partially or completely be aligned to a controllable electromagnetic energy source aligned to emit a non-biologically emitted electromagnetic energy in a proximity to the first focus and includes an opening configured to at least partially or completely allow the positioning of a portion of an animal body proximate with the second focus.

In an embodiment, the term “full ellipsoid” describes a structure that substantially encloses an ellipsoid or an ellipsoidally-shaped structure having one or more openings.

In an embodiment, the term “partial ellipsoid” in reference to a structure or substructure includes a structure or substructure comprising a lengthwise cross-section along the major axis of an ellipsoidally-shaped structure or substructure.

In an embodiment, the term “living body” refers to a human or any animal including domestic, marine, research, zoo, farm animals, fowl and sports animals, or pet animals, such as dogs, cats, cattle, horses, sheep, pigs, goats, rabbits, chicken, birds, fish, amphibian and reptile.

As used herein, the term “biological tissue” includes any portion of a living body or anatomy or morphology or a part of a physiology of a living body including intact or fragmented or sheared or isolated living or dead biological tissue or cells in culture or isolated cells in vitro or in vivo or ex vivo or individual colonies of microbial/eukaryotic cells or a single isolated cell. The biological tissue may include microbes, viruses, tissue isolates from living or non-living animals and/or plants.

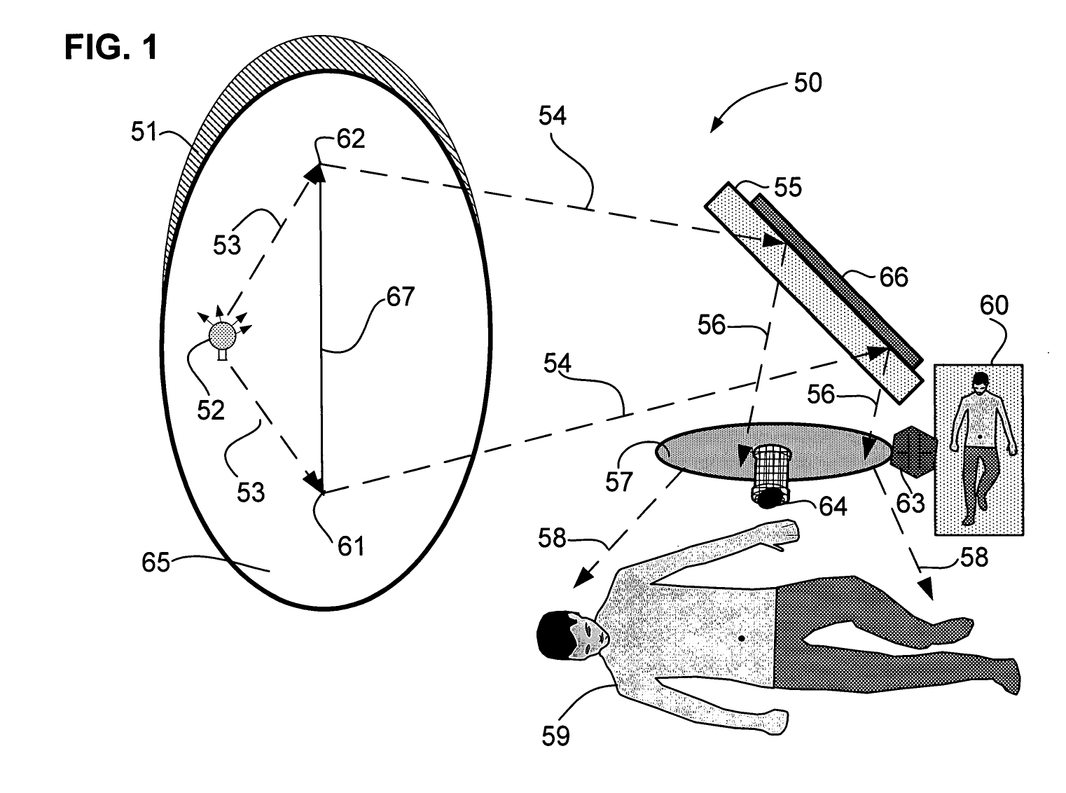

FIG. 1 illustrates an example of a medical device 50. The medical device may include at least one ellipsoidally-shaped reflector 51. In principle, the ellipsoidally-shaped reflector may produce two foci 61 and 62 that are coupled through a translational axis 67 of symmetry when light rays are reflected. In an embodiment, a controllable light source 52, which may be positioned in proximity to the reflector, may emit incident rays of light 53 (or other suitable electromagnetic radiation). The incident rays may form two foci 61 and 62 along the translational axis 67. In an embodiment, without limitations, the light source may emit UV, visible, near-infrared or other types of radiation. The incident rays of light 53 may be reflected off a reflector surface 65 and the reflected radiation 54 may be collected by a collector-mirror 55. In an embodiment, the collector-mirror may include one or more different types of mirrors such as, a concave mirror, a convex mirror, a plane mirror, a spherical mirror, a birefringence mirror etc. In an embodiment, one or more mirrors may be placed in a manner so as to provide a suitable amount and angle of reflection depending on a purpose to be achieved. This may include, but is not limited to, collection of light or radiation passing via the foci 61, 62 of the ellipsoid-shaped reflector 51 and directing the collected light to a biological tissue in a living body 59. In an additional or alternative embodiment, at least one mirror-cooling device 66 may be included in a mirror assembly system. Mirror-cooling devices are optional devices that may provide thermal control and stable isothermal performance of mirrors. Mirror-cooling devices may include, but are not limited to, back-plate cooling devices, liquid-cooled contact plates with, for example, glycol-water mixture flows, air-flushing fans, radiation plates, air-jet arrays and combinations thereof. For example, U.S. Pat. Nos. 4,606,620, 6,633,693 and 7,264,363, which are incorporated herein by reference, disclose mirrors and cooling devices. In an embodiment, reflected radiation 56 from the collector-mirror may pass through one or more lenses 57 or a plurality of beam-splitters 63. Typical lens systems may include without limitation, condenser lenses, double condenser systems or illumination systems, which may focus or telescope a light beam or expand a light beam 58 on to a living body/patient 59. Additionally or alternatively, the beam-splitters 63 may be coupled to a lens system to provide an adjustable beam cross-section. Furthermore, the beam splitters may be used for splitting light and directing a part of it into another device, such as an image receiver 60. In alternative embodiments, a plurality of beam splitters may be included in a system of interferometers and/or polarizers. An embodiment may include without limitation, a plurality of polarizers that may be configured to function in conjunction with beam splitters. Furthermore, beam splitters may divide an input polarized beam into a first and a second part with a dividing beam ratio, which may be continually controllable. In some embodiments, the polarizers polarize electromagnetic radiation either in linear, circular or elliptical polarization modes. An optical system in a medical device may include, without limitations, at least one monochromator 64 for selecting appropriate wavelength bands for treatment and/or for imaging. Imaging techniques may include, without limitations, autofluorescence detection and diagnosis of normal versus diseased biological tissues. In an embodiment, the medical device may include an image acquisition device, such as a camera or a sensor. Cameras generally may include, among other things, CCDs, two-dimensional array detectors, avalanche CCD photodetectors, photomultipliers, image contrast enhancers, near-infrared autofluorescence detectors, and photodiodes capable of point by point scanning.

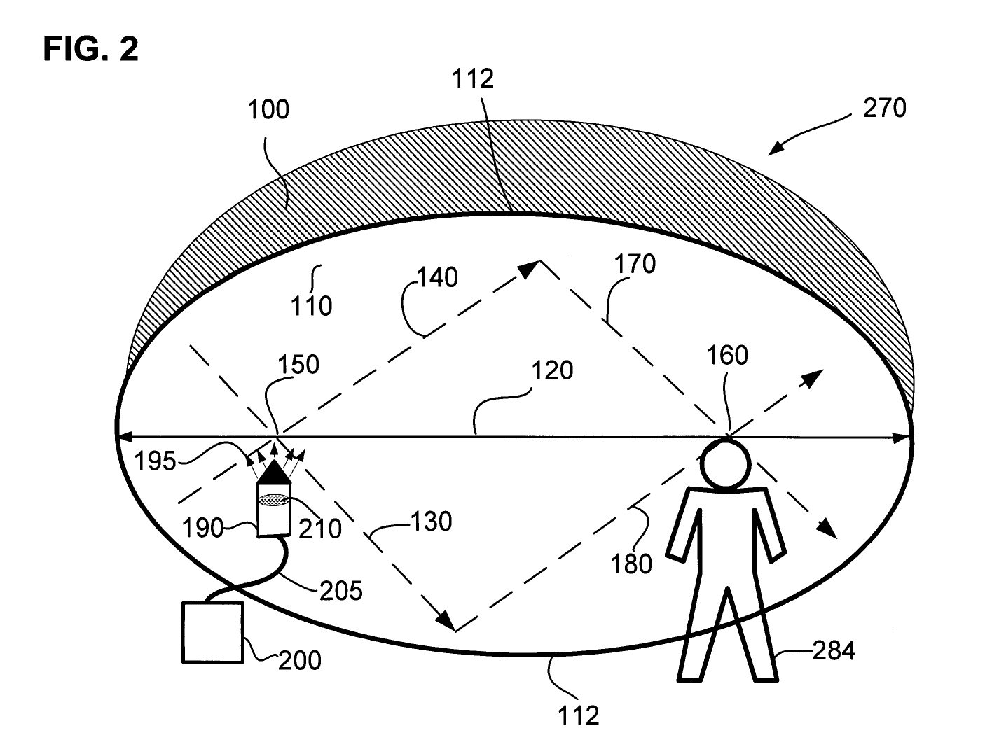

FIG. 2 illustrates an alternative embodiment of a medical device 270. The medical device comprises an ellipsoidally-shaped reflector 100 having a first focus 150 and a second focus 160, and providing a translational coupling 120 of non-biological electromagnetic energy 195 from the first focus to the second focus. The medical device also includes a controllable electromagnetic energy source 200 aligned to emit a non-biologically emitted electromagnetic energy 195 in a proximity to the first focus. In another embodiment, incident electromagnetic rays 130 and 140 pass through the first focus and reflect off the reflective surface 110 and form reflected rays 170 and 180, respectively, and converge at the second focus.

In a further embodiment, the ellipsoidally-shaped reflector 100 includes an opening 112 configured to allow positioning of a biological tissue of a living body 284 proximate with the second focus 160.

In another embodiment, at least of a portion of the reflector includes at least one of a metal, a dielectric, a liquid, a multilayer, a crystal, and/or a Bragg reflector.

In another embodiment, the non-biologically emitted electromagnetic energy 195 includes at least one of a visible light, a laser energy, an ultraviolet energy, an infrared energy, an X-ray and/or a microwave. In yet another embodiment, the electromagnetic energy emitter 190 is coupled 205 to an electromagnetic energy source 200 configured for controlled delivery of the non-biological electromagnetic energy 195. In another embodiment, the controllable electromagnetic energy source includes at least one of a plano-convex lens 210, a meniscus lens, a cylindrical lens, a parabolic lens, an acrylic lens, a glass lens, a quartz lens, a Fresnel lens, a Pendry lens, a band pass filter, a polarizer, a dichroic material, a monochromator and/or a collimator. In another embodiment, the controllable electromagnetic energy source regulates at least a characteristic of the non-biologically emitted electromagnetic energy. In a further embodiment, the at least a characteristic of the non-biologically emitted electromagnetic energy includes at least one of a wavelength, a frequency, an amplitude, a phase, a polarization and/or a bandwidth.

U.S. patent application Ser. Nos. 11/515,412 and 11/731,788, entitled “Electromagnetic Device and Method”, which are incorporated herein by reference in their entirety, discloses medical devices.

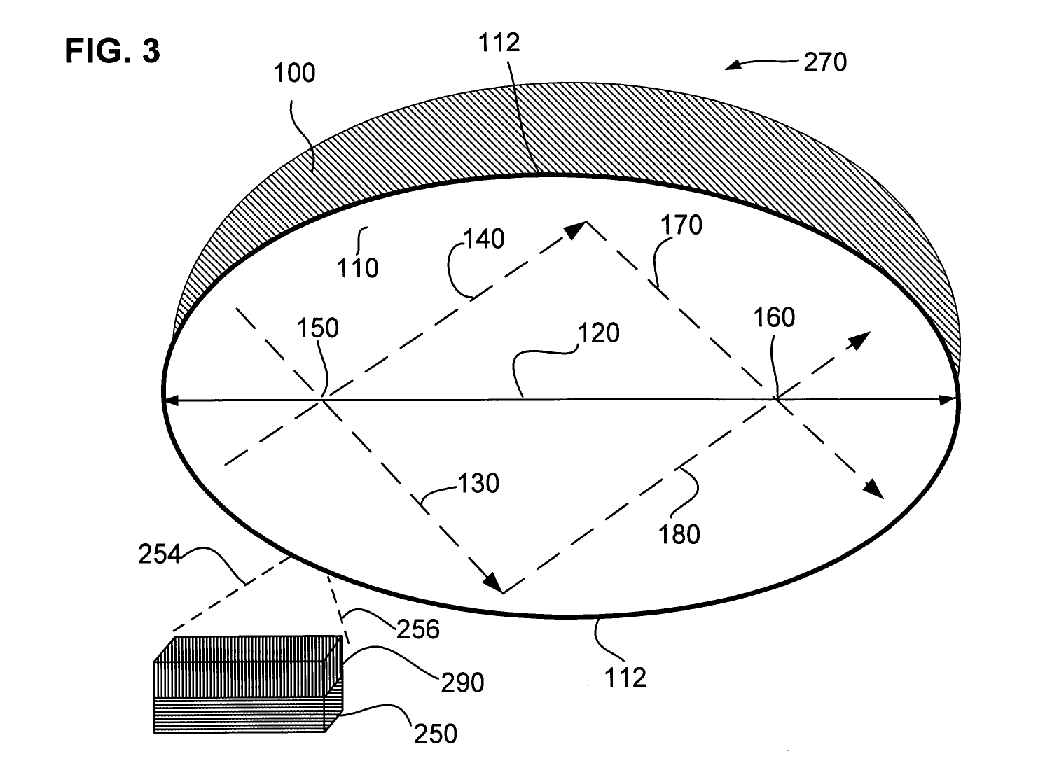

FIG. 3 shows an embodiment of a medical device 270 that includes a conductor 250 and a dielectric material 290. In an embodiment, an exploded view 254, 256 depicts the conductor and the dielectric material. In yet another embodiment, the conductor includes at least one of an aluminum, a tin, a stainless steel, a silver, a gold, a copper, an iron, a carbon, an iridium, an indium, a lead, a magnesium, a nickel, a nichrome, a palladium, a rhodium, a silver, a tantalum, a titanium, a tungsten, a zinc, a platinum, and/or a zirconium. In other embodiments, the dielectric material includes a rubber, a plastic, a porcelain, a ceramic, a mica, a glass, a plastics, a metal oxide, a perfect vacuum, a dry air, a pure dry gas such as helium and/or nitrogen.

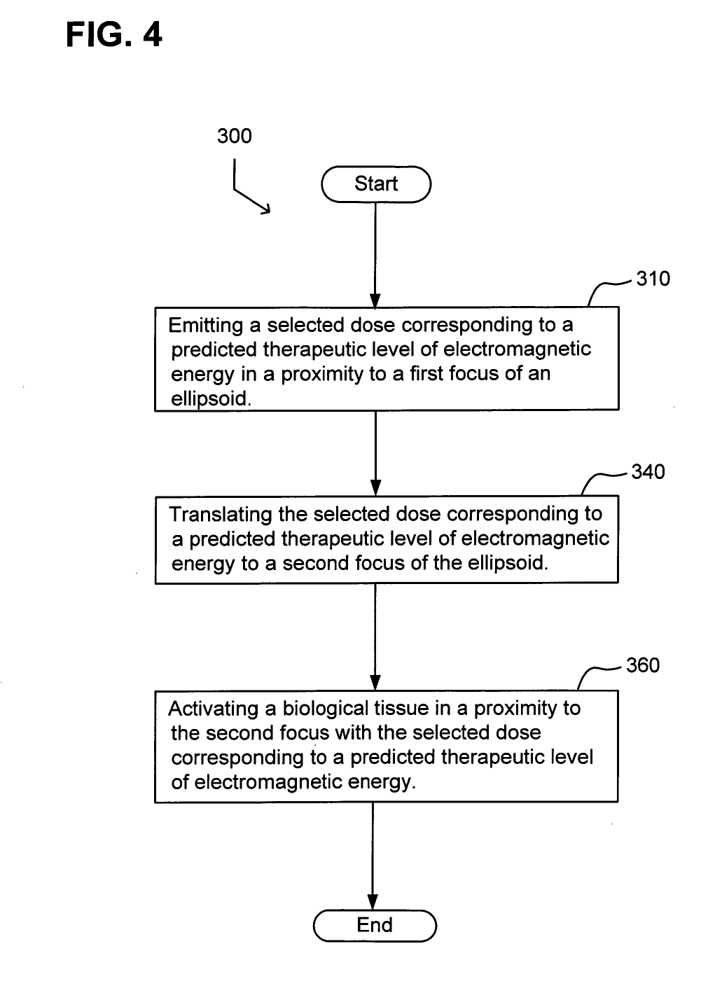

FIG. 4 illustrates an example of an operational flow 300 in which embodiments may be implemented. After a start operation, the operational flow moves to a radiating operation 310. The radiating operation emits a selected dose corresponding to a predicted therapeutic level of electromagnetic energy in a proximity to a first focus of an ellipsoid. A transposing operation 340 translates the selected dose corresponding to a predicted therapeutic level of electromagnetic energy to a second focus of the ellipsoid. An irradiation operation 360 activates a biological tissue in a proximity to the second focus with the selected dose corresponding to a predicted therapeutic level of electromagnetic energy. The operational flow moves to a stop operation.

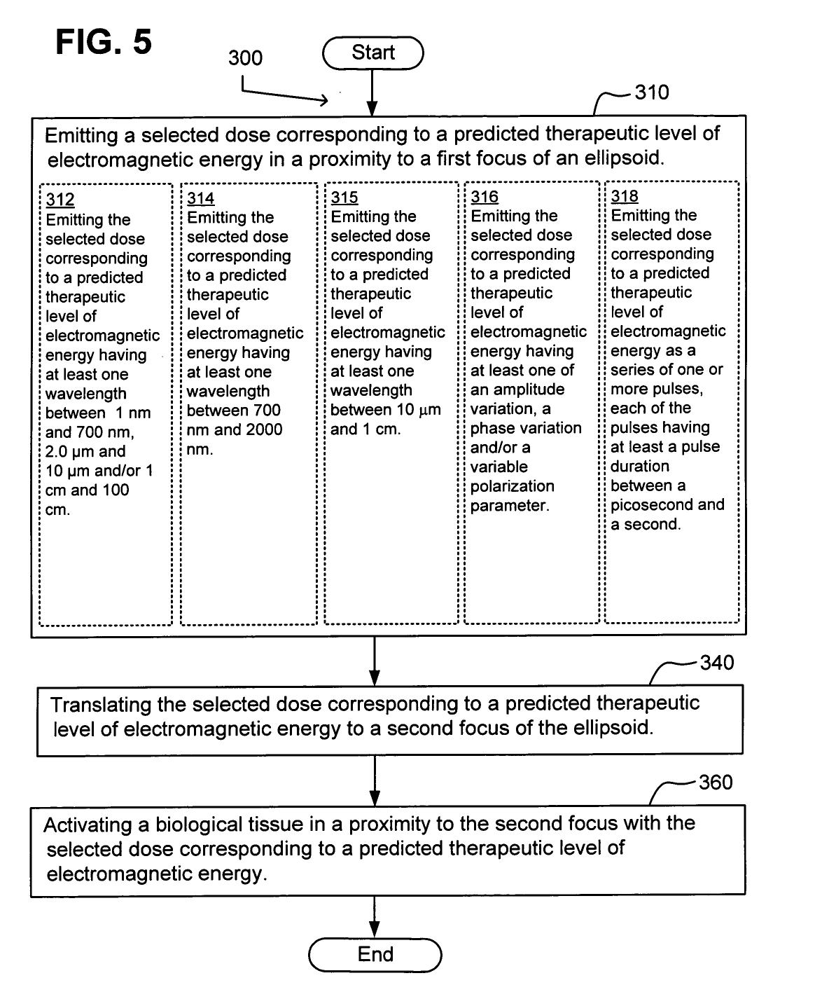

FIG. 5 illustrates an alternative embodiment of an operational flow 300 of FIG. 4. The radiating operation 310 may include at least one additional operation. The at least one additional operation may include an operation 312, an operation 314, an operation 315, an operation 316 and/or an operation 318. The operation 312 emits the selected dose corresponding to a predicted therapeutic level of electromagnetic energy having at least one wavelength between 1 nm and 700 nm, 2.0 μm and 10 μm and/or 1 cm and 100 cm. The operation 314 emits the selected dose corresponding to a predicted therapeutic level of electromagnetic energy having at least one wavelength between 700 and 2000 nm. The operation 315 emits the selected dose corresponding to a predicted therapeutic level of electromagnetic energy having at least one wavelength between 10 μm and 1 cm. The operation 316 emits the selected dose corresponding to a predicted therapeutic level of electromagnetic energy having at least one of an amplitude variation, a phase variation and/or a variable polarization parameter. The operation 318 emits the selected dose corresponding to a predicted therapeutic level of electromagnetic energy as a series of one or more pulses, each of the pulses having at least a pulse duration between a picosecond and a second.

In another embodiment, the wavelength of a selected dose corresponding to a predicted therapeutic level of electromagnetic energy includes at least the following wavelength ranges: from 1 nm to 10 nm; from 10 nm to 100 nm; from 100 nm to 700 nm; from 700 nm to 800 nm; from 800 nm to 900 nm; from 900 nm to 1000 nm; from 1000 nm to 1300 nm; from 1300 nm to 1700 nm; from 1700 nm to 2000 nm; from 2 μm to 3 μm; 3 μm to 5 μm; from 5 μm to 10 μm; 10 μm to 20 μm; from 20 μm to 30 μm; from 30 μm to 40 μm; from 40 μm to 50 μm; from 50 μm to 100 μm and from 100 μm to 1000 μm.

In other embodiments, the wavelength of a selected dose corresponding to a predicted therapeutic level of electromagnetic energy includes at least the following wavelength ranges: 0.1 cm to 0.5 cm; from 0.5 cm to 1 cm; from 1 cm to 5 cm; from 5 cm to 10 cm; from 10 cm to 20 cm; from 20 cm to 30 cm; from 30 cm to 40; from 50 cm to 60 cm and from 60 to 100 cm.

One of skill in the art will appreciate that in some embodiments, ranges of wavelength, frequency, amplitude, phase, polarization and/or a bandwidth and/or combinations thereof for selected doses corresponding to predicted therapeutic levels of electromagnetic energy may be utilized for different biological tissues and/or for different types of conductors in the ellipsoidal shaped reflector.

In some embodiments, the phrase “a selected dose corresponding to a predicted therapeutic level” of non-biological electromagnetic energy includes an energy level that is intended for delivery at a portion of a biological tissue and/or cell(s) to achieve inter alia a palliative and/or curative and/or therapeutic treatment and/or maintain/achieve a desired result for an animal patient or human patient or a research subject.

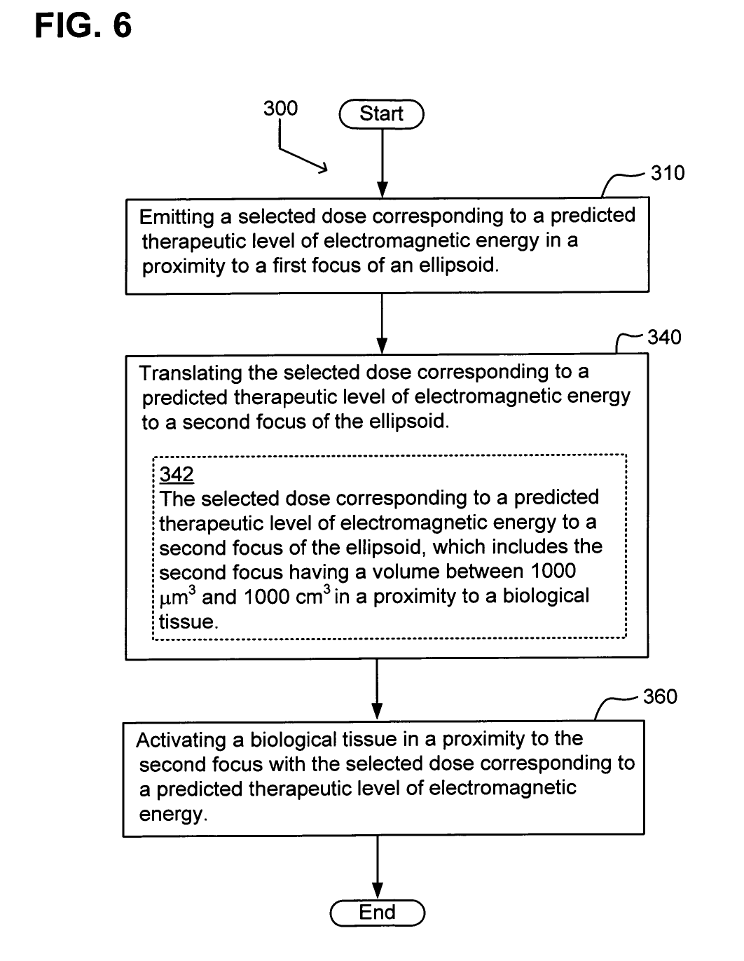

FIG. 6 illustrates an alternative embodiment of an example of operational flow 300 of FIG. 4. The transposing operation 340 may include at least one additional operation. The at least one additional operation may include an operation 342. The operation 342 translates the selected dose corresponding to a predicted therapeutic level of electromagnetic energy to a second focus of the ellipsoid, which includes the second focus having a volume between 1000 μm³ and 1000 cm³ in a proximity to a biological tissue.

In other embodiments, the second focus has a volume that includes at least the following ranges: from 1000 μm³ to 10,000 μm³; from 0.01 mm³ to 0.1 mm³; from 0.1 mm³ to 0.5 mm³; from 0.5 mm³ to 0.7 mm³; from 0.7 mm³ to 0.9 mm³; from 0.9 mm³ to 1.1 mm³; from 1.1 mm³ to 1.3 mm³; from 1.3 mm³ to 1.5 mm³; 1.5 mm³ to 2.0 mm³; from 2 mm³ to 5 cm³; from 5 cm³ to 10 cm³; from 10 cm³ to 100 cm³ and/or from 100 cm³ to 1000 cm³.

One of skill in the art will appreciate that in some embodiments, a single second focal volume and/or a single power and/or a multiplicity of ranges of focal volumes and/or power and/or combinations thereof may be utilized for different biological tissues and/or for different types of conductors in the ellipsoidal shaped reflector.

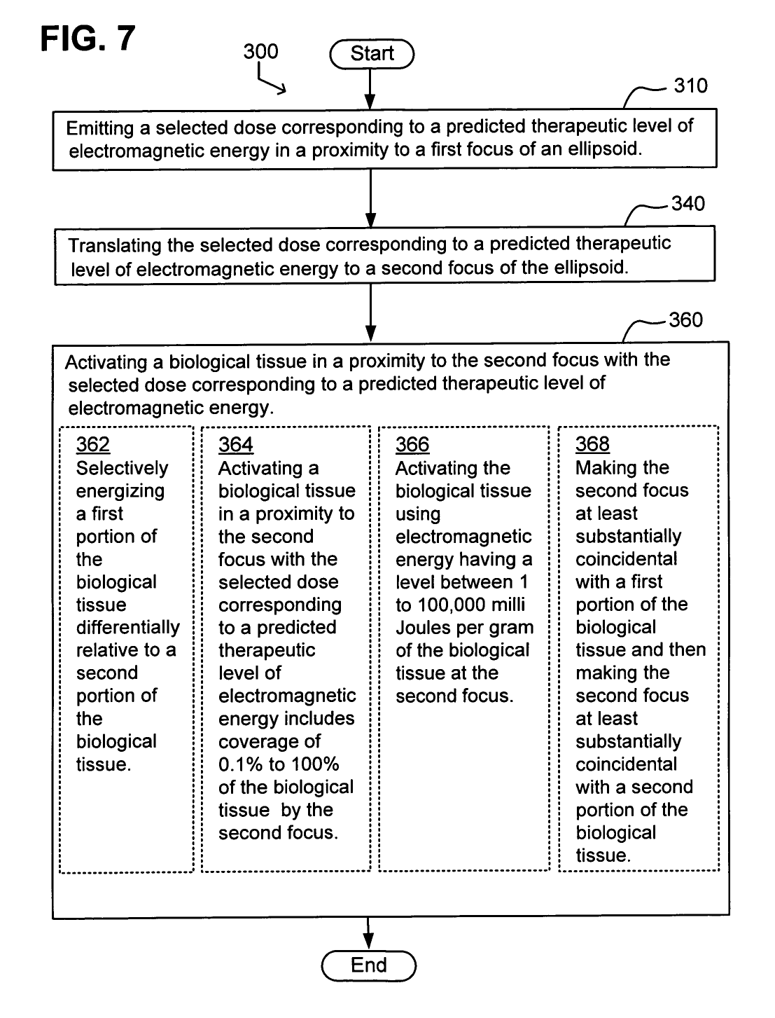

FIG. 7 illustrates an alternative embodiment of an operational flow 300 of FIG. 4. The irradiation operation 360 may include at least one additional operation. The at least one additional operation may include an operation 362, an operation 364, an operation 366 and/or an operation 368. The operation 362 selectively energizes a first portion of the biological tissue differentially relative to a second portion of the biological tissue. The activating operation 364 achieves a coverage of 0.1% to 100% of the biological tissue with the second focus. The activation operation 366 activates the biological tissue using electromagnetic energy having a level between 1 to 100,000 milli Joules per gram of the biological tissue at the second focus. The activation operation 368 makes the second focus at least substantially coincidental with a first portion of the biological tissue and then making the second focus at least substantially coincidental with a second portion of the biological tissue.

In some embodiments the coverage of second focus with the selected dose corresponding to a predicted therapeutic level of electromagnetic energy includes coverage of approximately from 0.1% to 1%; from 1% to 10%; from 10% to 20%; from 20% to 30%; from 30% to 40%; from 40% to 50%; from 50% to 60%; from 60% to 70%; from 70% to 80%; from 80% to 90%; and from 90% to 100%. One of skill in the art will recognize that the extent of coverage by second focus with the selected dose corresponding to a predicted therapeutic level of electromagnetic energy depends on the size, depth and shape of the target biological tissue. One of skill in the art will also recognize that the extent of coverage by second focus with the selected dose corresponding to a predicted therapeutic level of electromagnetic energy is related to the size of the focal area of the second focus vis-a-vis the size of the target biological tissue. For instance, if the size of the target biological tissue is larger than the second focal area then the effective coverage by second focus with the selected dose corresponding to a predicted therapeutic level of electromagnetic energy will be less than 100% of the target biological tissue. One of skill in the art will recognize that in this case a plurality of activation regimes at second focus with the selected dose corresponding to a predicted therapeutic level of electromagnetic energy will be necessary to achieve 100% coverage of the target biological tissue. In this case, activation regimes can be adjusted to cover the target biological tissue in increments of less than 100% coverage at one time and repeating activation regimes at second focus multiple times until the desired level of coverage is achieved.

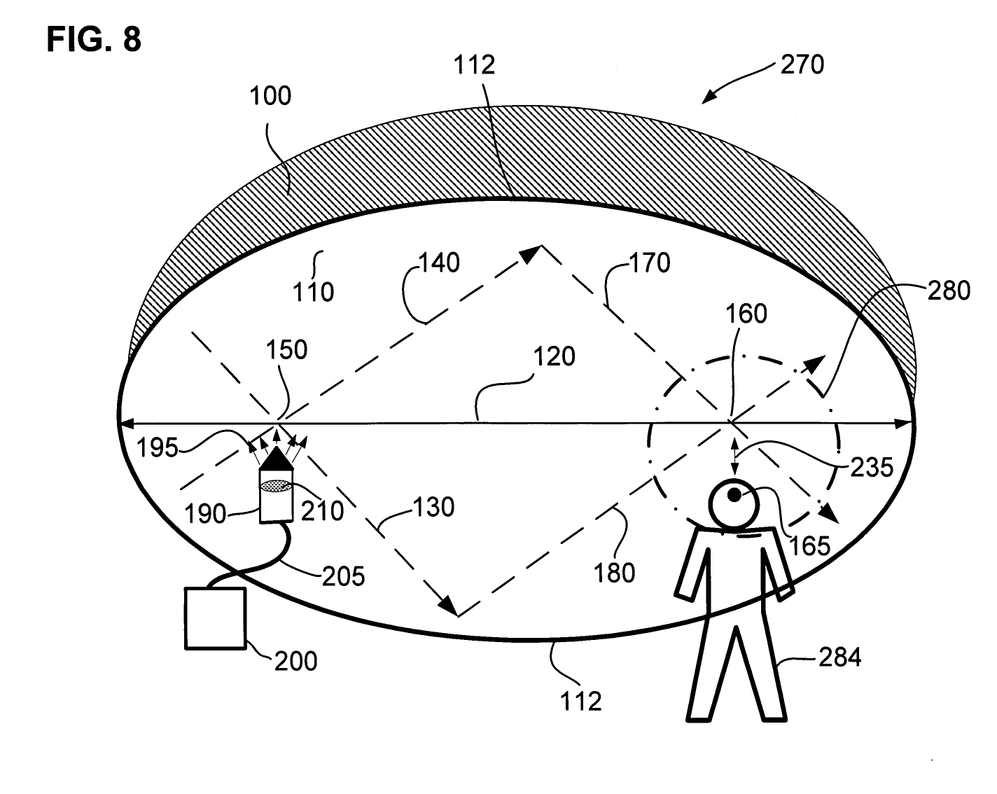

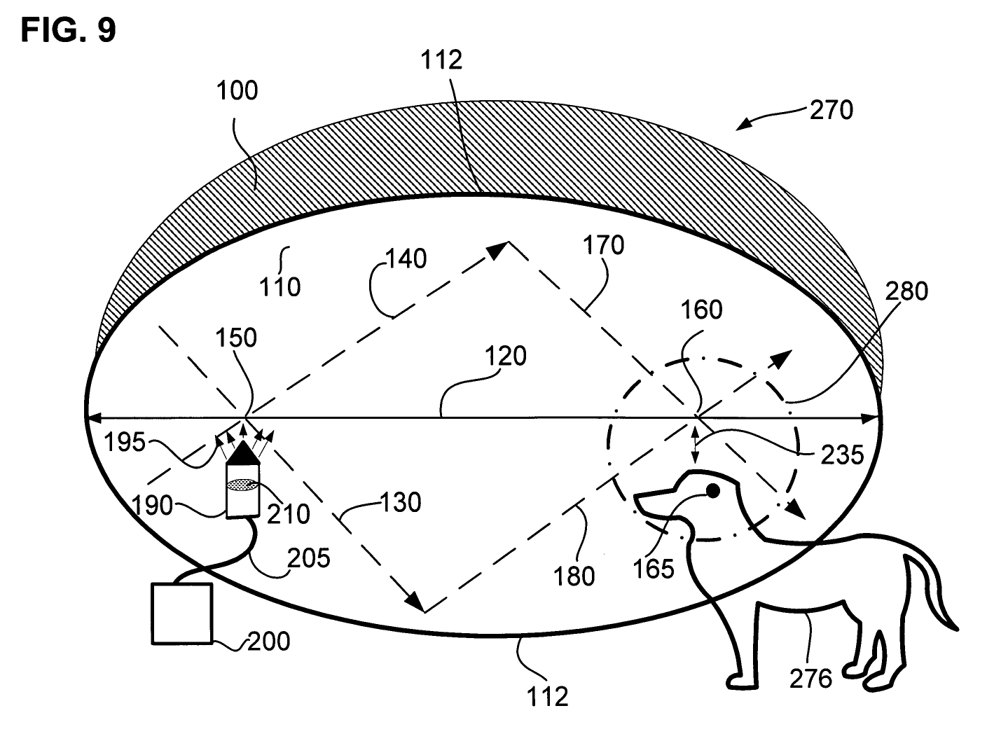

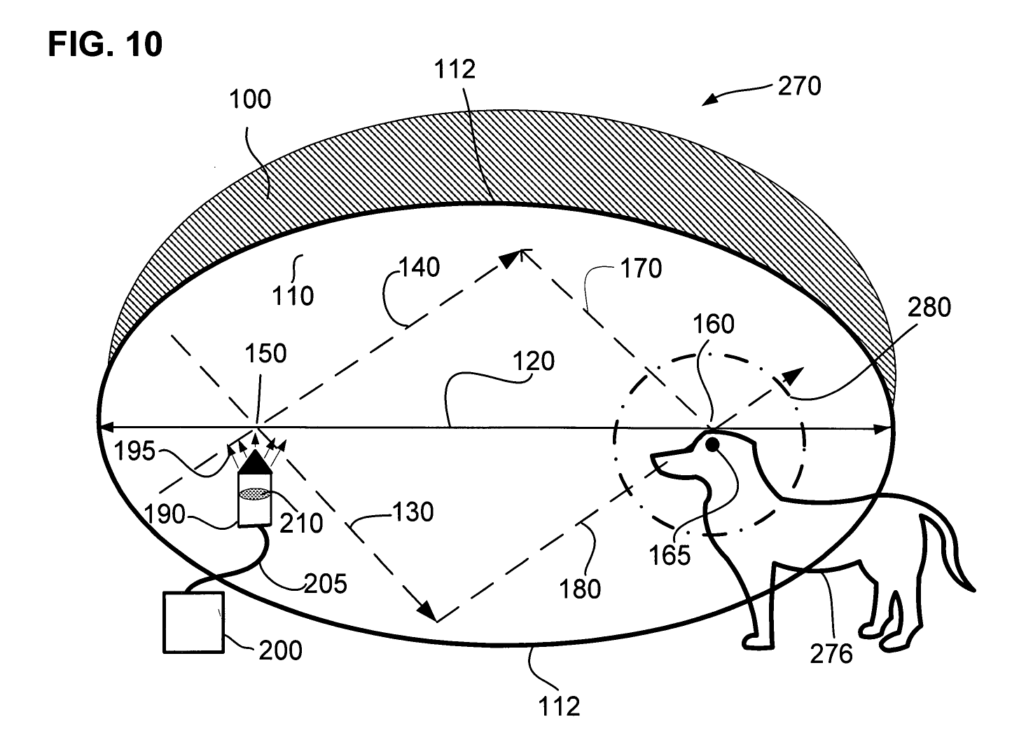

FIGS. 8 through 11 schematically illustrate a medical device 270 in which an embodiment of operational flow 300 of FIG. 4 may be implemented. FIGS. 8-11 depict alternative embodiments, which may be implemented in a zone of activation 280. Biological tissue 165 from a human body 284 may be activated in the zone of activation. The medical device 270 provides irradiation either in a proximity 235 (FIGS. 8 and 9) to the second focus 160 or irradiation in substantial coincidence (FIG. 10) with the second focus. One of skill in the art will appreciate that the proximity of the second focus may be achieved by either moving the ellipsoid reflector or moving the living body and/or moving both depending on a required therapeutic dosage and/or dimensions of the living body.

FIGS. 9 and 10 depict a dog 276 as the subject/patient for treatment. In FIG. 9 the biological tissue 165 is in a proximity 235 to the second focus 160 whereas in FIG. 9 the biological tissue is in substantial coincidence with the second focus. In principle, any animal may be substituted for the dog in FIGS. 9 and 10.

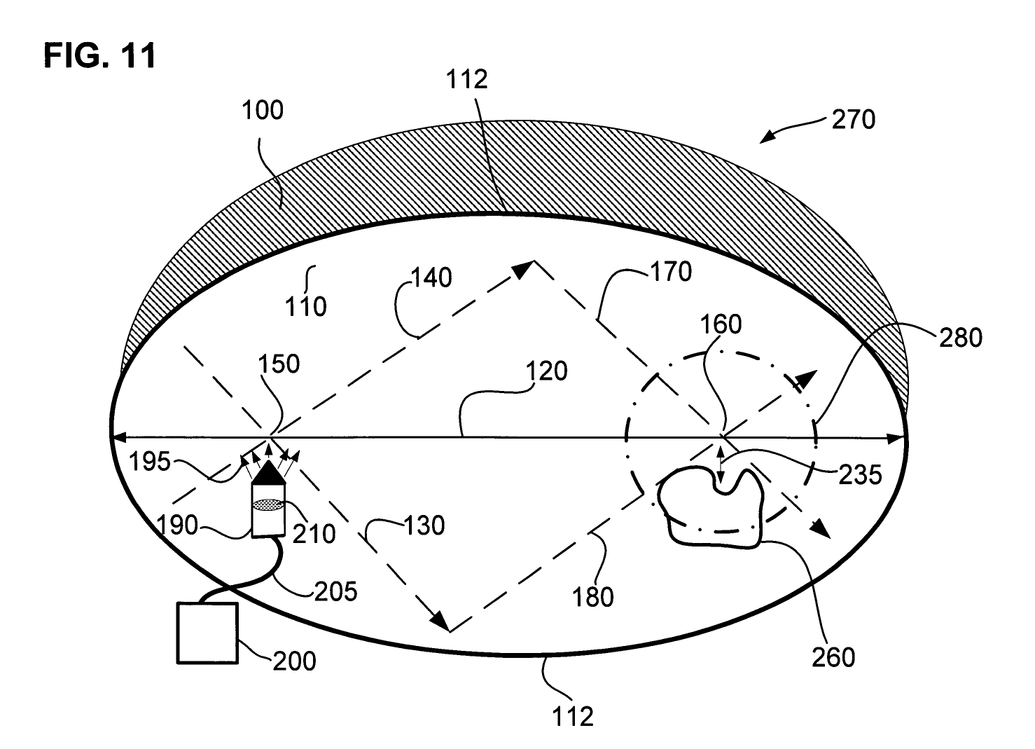

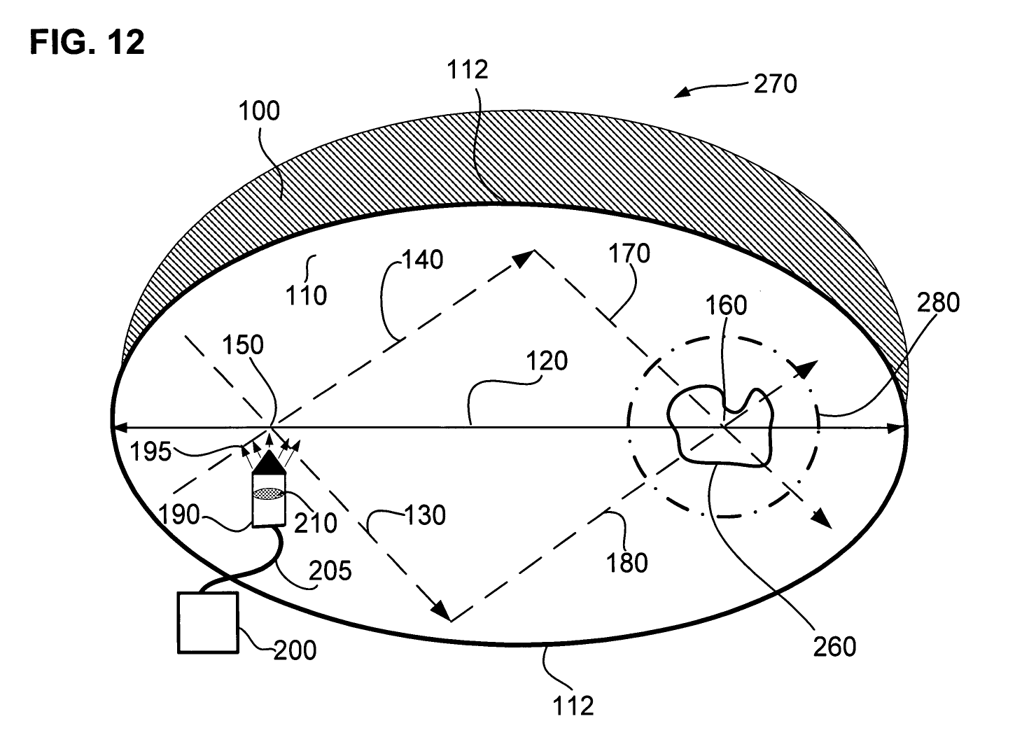

FIGS. 11 and 12 depict further embodiments, which may be implemented in the medical device 270. Herein, isolated biological tissue 260 is treated in the zone of activation 280 either in a proximity 235 (FIG. 11) to the second focus 160 or treated in substantial coincidence (FIG. 12) with the second focus 160.

One of skill in the art will appreciate that differentially irradiating different portions of the biological tissue may include changing wavelength, amplitude, phase, polarization, power, focal volume, focal depth and/or focal area of the second focus.

Returning to FIGS. 11 and 12, some alternative embodiments that may be implemented in the medical device 270 for activation of the biological tissue 260 may include, inter alia, administration of a plurality of temporally spaced irradiations of the biological tissue. For example, a portion of the biological tissue may be activated first for certain duration of time followed by an interval of non-activation that is followed by a second activation period followed by a non-activation period and then by a third activation period, so on and so forth.

In certain embodiments, the temporal activation of the biological tissue 260 comprises activation of a first portion (not shown) of the biological tissue which is immediately followed by activation of a second portion (not shown) of biological tissue that is immediately followed activation of a third portion (not shown) of biological tissue, so on and so forth, until complete activation/coverage is achieved for all required portions of biological tissue and/or animal body.

A person of skill in the art will recognize that the selected dose corresponding to a predicted therapeutic level of electromagnetic energy for activation of different portions of a biological tissue may be different depending on palliative or therapeutic or other purposeful desired result to be achieved. Likewise, the selected dose corresponding to a predicted therapeutic level of electromagnetic energy required for partial and/or complete activation of a given biological tissue from a certain origin (animal or plant or microbial) may be different compared to the selected dose required for activation of a biological tissue of a different origin. In a similar vein, one of skill in the art will realize that the selected dose corresponding to a predicted therapeutic level of electromagnetic energy required for partial or complete activation of biological tissues from different parts of the same animal (or plant or microbial cells) may require a different the selected dose for obtaining a desired level of activation. For example, treatment regimes for brain tumors may differ based on the nature, origin, location (e.g. depth of location), shape and size of the tumors.

In an embodiment, an electromagnetic energy-activated biological tissue may include an electromagnetic energy-mediated activated biological tissue. The electromagnetic energy-mediated activated biological tissue may be activated to a state of a necrosis, an induced apoptosis, a non-lethal metabolic physiological alteration, and/or an enhancement of tissue function.

In some embodiments, the term “activation” includes achieving a desired purpose with electromagnetic energy at an anatomical site or area of a living body and/or biological tissue that is intended to receive the radiation as prescribed in a dosage regime.

An alternative embodiment may include a combination of an electromagnetic energy, a biological tissue, and an endogenous or an exogenous pharmacological agent or drug. In combination, the pharmacological agent or drug may be activated in vivo to achieve a conformational or a functional alteration with respect to the biological tissue. One skilled in the art will recognize that the combination of the electromagnetic energy, the biological tissue, and the agent or drug may be used to achieve a focalized activation state of a locally-distributed or a systemically-distributed agent or drug.

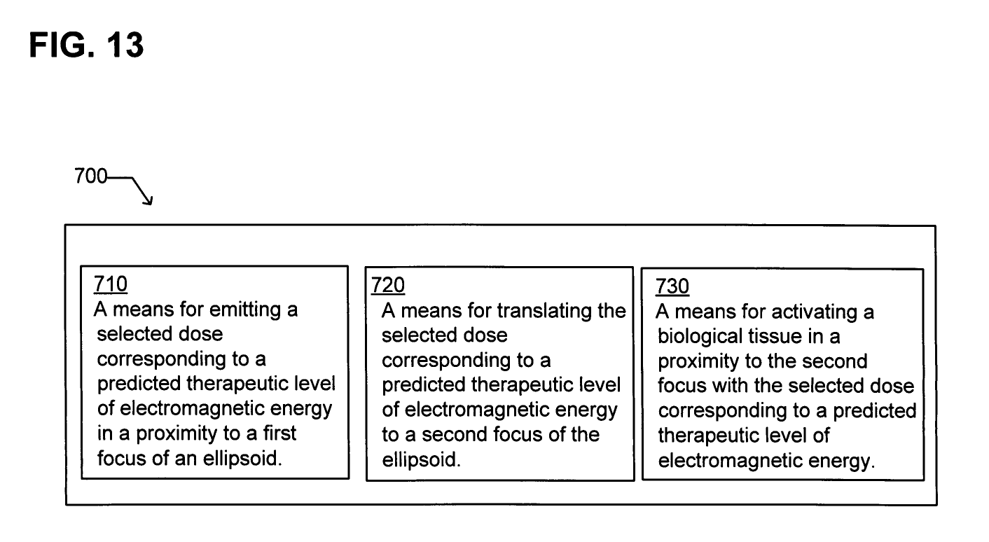

FIG. 13 illustrates an example of a medical device 700 that may be used to implement embodiments. The device includes a means 710 for emitting a selected dose corresponding to a predicted therapeutic level of electromagnetic energy in a proximity to a first focus of an ellipsoid. The medical device also includes a means for translating 720 the selected dose corresponding to a predicted therapeutic level of electromagnetic energy to a second focus of the ellipsoid. The medical device further includes a means for activating 730 a biological tissue in a proximity to the second focus with the selected dose corresponding to a predicted therapeutic level of electromagnetic energy.

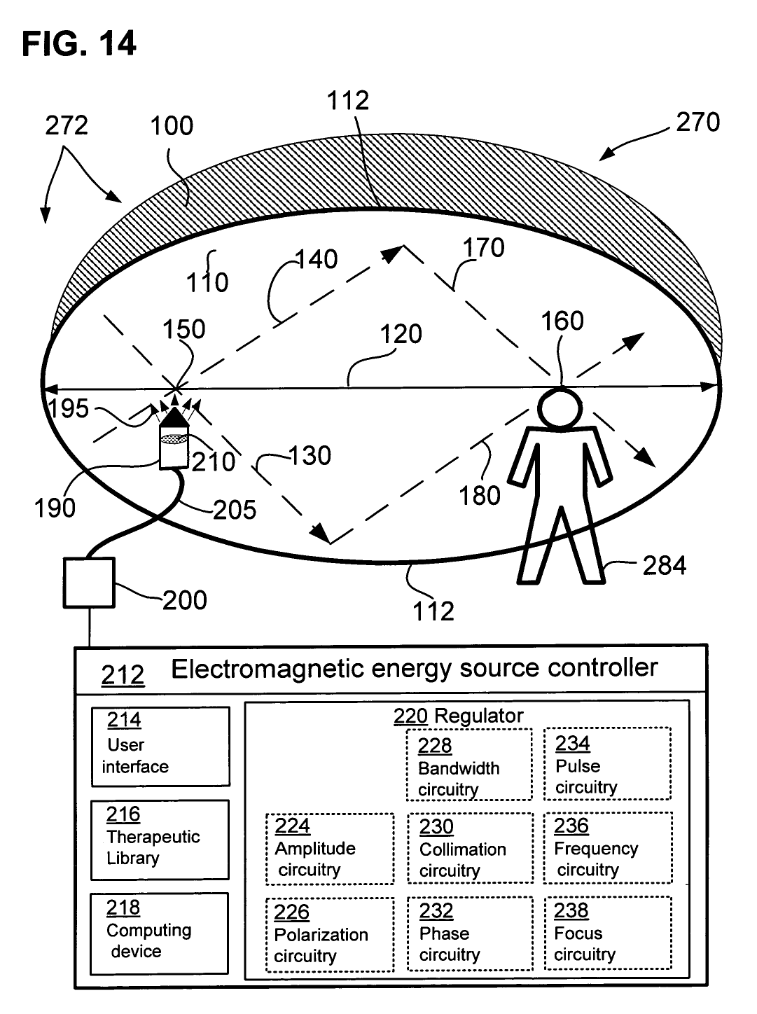

FIG. 14 illustrates an example of a system 272 that may be used to implement embodiments. An embodiment of the system includes an ellipsoidally-shaped reflector 100 having a first focus 150 and a second focus 160, shaped to provide a translational coupling 120 of non-biological electromagnetic energy 195 from the first focus 150 to the second focus 160. A further embodiment of the system includes a controllable electromagnetic energy source 200 aligned to emit a non-biologically emitted electromagnetic energy in a proximity to the first focus. In yet another embodiment, the system includes an electromagnetic energy source controller 212 that is coupled to the energy source and having a regulator 220. In an embodiment, the electromagnetic energy source controller includes a user interface 214. In a further embodiment, the electromagnetic energy source controller includes a therapeutic library 216 that includes at least one of a treatment regime. In another embodiment, the electromagnetic energy source controller includes a computing device 218 that includes at least one computer. In a further embodiment, the regulator includes an amplitude-regulating electrical circuitry 224 configured to govern at least one amplitude emitted by the electromagnetic energy source. In another embodiment, the regulator includes a polarization-regulating electrical circuitry 226 configured to govern at least one polarization state emitted by the electromagnetic energy source. In yet another embodiment, the regulator includes a bandwidth-regulating electrical circuitry 228 configured to govern at least one bandwidth emitted by the electromagnetic energy source. In a further embodiment, the regulator includes a collimation-regulating electrical circuitry 230 configured to govern at least one collimation filter of the electromagnetic energy source. In an embodiment, the regulator includes a phase-regulating electrical circuitry 232 configured to govern at least one phase shift emitted by the electromagnetic energy source. In another embodiment, the regulator includes a pulse-regulating electrical circuitry 234 configured to govern at least one pulse emitted by the electromagnetic energy source. In a further embodiment, the regulator includes a frequency-regulating electrical circuitry 236 configured to govern at least one frequency emitted by the electromagnetic energy source. In an embodiment, the regulator includes a focus-regulating electrical circuitry 238 configured to govern at least one focal area of the electromagnetic energy.

Embodiments may be adapted for use in scanning and imaging devices working in conjunction with charge coupled devices. For instance, the non-biological electromagnetic energy source may be tuned to emit a specific bandwidth or wavelength of radiation to scan biological tissues, vascular structures, brain and/or other internal organs for scanning-imaging purposes. Embodiments may also be used in conjunction with fluorescence spectroscopy and/or diffuse reflectance spectroscopy/scattering technologies and/or optical spectroscopy and/or magnetic resonance spectroscopy of biological tissues. Likewise, embodiments may be adapted for skin exfoliation, skin rejuvenation treatments in conjunction with appropriate chemicals, for photodynamic therapy, collagen regenerative therapy, clearing blemishes, ex vivo blood purification therapy and ex situ imaging design. Another potential application is the adaptation of embodiments disclosed herein to imaging of tumors, biological tissue and/or whole bodies. Thus the embodiments may be adapted to operate in detection mode, demarcation mode, scanning mode and/or treatment mode.

The foregoing detailed description has set forth various embodiments of the devices and/or processes via the use of flowcharts, diagrams, figures and/or examples. Insofar as such flowcharts, diagrams, figures and/or examples contain one or more functions and/or operations, it will be understood by those within the art that each function and/or operation within such flowchart, diagram, figure and/or example can be implemented, individually and/or collectively, by a wide range of any combination thereof.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

The herein described subject matter sometimes illustrates different components contained within, or connected with, different other components. It is to be understood that such depicted figures are merely exemplary, and that in fact many other figures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or systems in the fashion set forth herein, and thereafter use engineering practices to integrate such described devices and/or systems into electromagnetic radiation systems. That is, at least a portion of the devices and/or system described herein can be integrated into an electromagnetic radiation system via a reasonable amount of experimentation. Those having skill in the art will recognize that a typical electromagnetic radiation system generally includes one or more of a system unit housing, video display devices, memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position and/or velocity; control motors for moving/distorting various optical and non-optical components. One skilled in the art will recognize that a typical electromagnetic radiation system includes, but is not limited to, a variety of optical and non-optical components such as lenses, filters, focusers, mirrors, collimators, monochromators, optical beam splitters, optical beam shifters, polarizers; wavelength, frequency, bandwidth, and/or phase modulators and/or controllers; optical and/or non-optical radiation emitters such as pulse and/or continuous lasers, arcs, lamps, LEDs, linear and/or nonlinear optical devices, radioactive element-based sources, micro wave emitters, ultra sonic and/or sonic emitters. A typical electromagnetic radiation system and/or an improvement thereof may be implemented utilizing one or more suitable commercially available components, including but not limited to the above-listed components.

While particular aspects of the present subject matter described herein have been shown and described, it will be apparent to those skilled in the art that, based upon the embodiments herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A medical device, comprising:
an ellipsoidally-shaped reflector having a first focus and a second focus, and providing a translational coupling of electromagnetic energy from the first focus to the second focus; and
a controllable electromagnetic energy source aligned to emit a non-biologically emitted electromagnetic energy in a proximity to the first focus.

2. The device of claim 1, wherein the ellipsoidally-shaped reflector includes an opening configured to allow positioning of a portion of a body proximate with the second focus.

3. The device of claim 1, wherein the ellipsoidally-shaped reflector includes a conductor.

4. The device of claim 3, wherein the conductor includes at least one of an aluminum, a tin, a stainless steel, a silver, a gold, a copper, an iron, a carbon, an iridium, an indium, a lead, a magnesium, a nickel, a nichrome, a palladium, a rhodium, a silver, a tantalum, a titanium, a tungsten, a zinc, a platinum and/or a zirconium.

5. The device of claim 1, wherein the ellipsoidally-shaped reflector includes a dielectric material.

6. The device of claim 5, wherein the dielectric material includes a rubber, a plastic, a porcelain, a ceramic, a mica, a glass, a metal oxide, a perfect vacuum, a dry air, a pure dry gas, a helium and/or a nitrogen.

7. The device of claim 1, wherein the non-biologically emitted electromagnetic energy includes at least one of a visible light, a laser energy, an ultraviolet energy, an infrared energy, an X-ray, and/or a microwave.

8. The device of claim 1, wherein the controllable electromagnetic energy source includes a computer configured to control delivery of the non-biologically emitted electromagnetic energy.

9. The device of claim 1, wherein the controllable electromagnetic energy source includes at least one of a plano-convex lens, a meniscus lens, a cylindrical lens, a parabolic lens, an acrylic lens, a glass lens, a quartz lens, a Fresnel lens, a Pendry lens, a band pass filter, a polarizer, a dichroic material, a monochromator and/or a collimator.

10. The device of claim 1, wherein the controllable electromagnetic energy source regulates at least a characteristic of the non-biologically emitted electromagnetic energy.

11. The device of claim 10, wherein the at least a characteristic of the non-biologically emitted electromagnetic energy includes at least one of a wavelength, a frequency, an amplitude, a phase, a polarization and/or a bandwidth.

12. The device of claim 1, further comprising a radiation-collector mirror.

13. The device of claim 12, wherein the radiation-collector mirror is a birefringence mirror.

14. The device of claim 12, wherein the radiation-collector mirror is a light collector-mirror.

15. The device of claim 14, wherein the light collector-mirror collects light from the second focus and directing collected light to a biological tissue.

16. The device of claim 12, wherein the radiation-collector mirror includes at least one mirror-cooling device.

17. The device of claim 1, further comprising a plurality of beam-splitters.

18. The device of claim 17, wherein at least one of the plurality of beam-splitters produces an adjustable beam cross-section.

19. The device of claim 1, further comprising a plurality of polarizers.

20. The device of claim 19, wherein the plurality of polarizers polarize electromagnetic radiation either in a linear, a circular or an elliptical polarization mode.

21. The device of claim 19, wherein at least one of the plurality of polarizers is configured to function in conjunction with a beam splitter.

22. The device of claim 21, wherein the beam splitter divides an input polarized beam into a first and a second part with a dividing beam ratio which is continually controllable.

23. The device of claim 1, further comprising a condenser lens system.

24. The device of claim 1, further comprising a monochromator.

25. The device of claim 1, further comprising an image receiver.
